(19) United States
(12) Patent Application Publication
(10) Pub. No.: US 2024/0197194 A1
(43) Pub. Date: Jun. 20, 2024

(54) BIOLOGICAL INFORMATION MEASUREMENT DEVICE, CONTROL METHOD FOR BIOLOGICAL INFORMATION MEASUREMENT DEVICE, AND PROGRAM

(71) Applicant: OMRON HEALTHCARE CO., LTD., Kyoto (JP)

(72) Inventors: Takahiro HAMAGUCHI, Kyoto (JP); Yuka TANABE, Kyoto (JP); Kentaro MORI, Kyoto (JP); Takeshi KUBO, Kyoto (JP); Yuki TAKANO, Kyoto (JP); Yasuo ASANO, Kyoto (JP); Rika KATO, Kyoto (JP)

(73) Assignee: OMRON HEALTHCARE CO., LTD., Kyoto (JP)

(21) Appl. No.: 18/592,582

(22) Filed: Mar. 1, 2024

Related U.S. Application Data
(63) Continuation of application No. PCT/JP2023/004245, filed on Feb. 8, 2023.

(30) Foreign Application Priority Data
Jun. 23, 2022 (JP) 2022-101233

(51) Int. Cl. A61B 5/024; A61B 5/00; A61B 5/0245

(52) U.S. Cl. CPC A61B 5/02438; A61B 5/0245; A61B 5/681; A61B 5/684; A61B 5/7475; A61B 2560/0468; A61B 2562/16

(57) ABSTRACT

This device is used by being worn on the wrist of the human body, said device comprising: a blood pressure measuring unit; an electrocardiographic waveform measuring unit, which is provided with a plurality of electrodes and is for measuring electrocardiographic waveforms of the human body; an electrode contact state detecting unit for detecting a contact state of the human body with the electrodes; a position detecting unit for detecting the position of the device; a control unit; an input unit for receiving a measurement start instruction; a first propriety determination unit that assesses whether the device is positioned at a height within a prescribed range; a second propriety determination unit that assesses whether the human body is in stable contact with the plurality of electrodes; and a combined measurement control unit that performs control for executing combined measurements of the blood pressure and the electrocardiographic waveform of the human body.

### CROSS-REFERENCE TO RELATED APPLICATIONS

This application is the U.S. national stage application filed pursuant to 35 U.S.C. 365(c) and 120 as a continuation of International Patent Application No. PCT/JP2023/004245, filed Feb. 8, 2023, which application claims priority to Japanese Patent Application No. 2022-101233, filed Jun. 23, 2022, which applications are incorporated herein by reference in their entireties.

### TECHNICAL FIELD

The present invention belongs to a technical field related to healthcare, and particularly relates to a biological information measurement device, a control method of a biological information measurement device, and a program.

### BACKGROUND ART

In recent years, it has been common for an individual to measure his/her body/health information (hereinafter, also referred to as biological information) such as a blood pressure value and an electrocardiographic waveform by using a measurement instrument on a daily basis and utilize the measurement result for health management. For this reason, there is an increasing demand for instruments focusing on portability, many portable measurement devices have been proposed, and a portable instrument capable of measuring both the blood pressure value and the electrocardiographic waveform has also been proposed (for example, Patent Literature 1 and the like).

Patent Literature 1 discloses a portable electrocardiogram measurement device having means for performing blood pressure measurement in an electrocardiographic waveform measurement device worn on a wrist of a human body by using a belt including an electrode. According to the present invention, by carrying the device, a user can obtain electrocardiographic waveform information indicating the electrical activity of a heart and measure the blood pressure at an arbitrary timing such as when the user feels a pain in a chest. In addition, it is also described that, when the device is worn on the wrist, the electrocardiographic waveform (and the blood pressure) can be measured by so-called IV induction only by placing, on the chest, a device body fixed to an arm, and the electrocardiographic waveform can be measured by I induction by attaching the device to one (right) arm and touching the electrode arranged on the device body with the other (left) hand. According to these measurement methods, it is not necessary to hold the device body with a hand, and unnecessary force is not applied, so that a signal with less noise caused by myoelectric potential or the like can be obtained.

### CITATION LIST

### Patent Literature

Patent Literature 1: JP 2007-195693 A

### SUMMARY OF INVENTION

By the way, according to the technique described in Patent Literature 1, when performing measurement using the device, in the case of IV induction, the user wears the device on the right wrist and then brings the electrode into contact with the chest, and presses the measurement start button of the device body with the left hand on which the device is not worn, to start the measurement. In addition, in the case of I induction, it is necessary to press the measurement start button with the left hand and then touch the electrode of the device body with the hand.

However, in such a measurement mode, there is a possibility that a state where a contact state with the electrode is not appropriate occurs after the measurement start button is pressed (that is, after the measurement is started), so that an unstable electrocardiogram may be recorded, or that particularly in the case of I induction, the measurement proceeds without touching the electrode. An explanatory diagram illustrating such a situation is illustrated in FIG. 10. In addition, in a case where the blood pressure measurement is performed together with the measurement of the electrocardiographic waveform, it is necessary to take a posture in which the device body (a place where the blood pressure measurement is performed) is arranged at the same height as the height of the heart, so that the user is forced to take the inconvenient posture, which makes the above problem more remarkable.

In view of the above problems, an object of the present invention is to provide a technique for accurately measuring blood pressure and an electrocardiographic waveform in a portable biological information measurement device capable of measuring the blood pressure and the electrocardiographic waveform.

In order to solve the above problem, the present invention adopts the following configuration. That is, provided is a biological information measurement device used by being worn on a wrist of a human body, the biological information measurement device including:
a blood pressure measurement unit configured to measure a blood pressure of the human body;
an input unit configured to receive an instruction to start blood pressure measurement of the human body;
an electrocardiographic waveform measurement unit which includes a plurality of electrodes and is configured to measure an electrocardiographic waveform of the human body;
an electrode contact state detection unit configured to detect a contact state of the human body with the plurality of electrodes;
a position detection unit configured to detect a position of the device; and
a controller which controls the electrocardiographic waveform measurement unit and the blood pressure measurement unit,
in which the control unit includes
   a first correctness determination unit which determines, on the basis of an output of the position detection unit, correctness as to whether or not a wrist of the human body on which the device is worn is positioned at a height within a first predetermined range after receiving an instruction to start blood pressure measurement of the human body via the input unit,
   a second correctness determination unit which determines, on the basis of an output of the electrode contact state detection unit, correctness as to whether or not the human body is stably in contact with the plurality of electrodes, and
   a collective measurement control unit which performs control to collectively execute blood pressure measurement of the human body by the blood pressure measurement unit and measurement of an electrocardiographic waveform of the human body by the electrocardiographic waveform measurement unit, on condition that at least a determination result of the second correctness determination unit indicates correctness.

Note that in the present specification, “measurement of an electrocardiographic waveform” unit recording of waveform data of an electrocardiographic signal. In addition, “collectively” includes simultaneous and parallel execution. In addition, examples of the above-described blood pressure measurement unit include a cuff, a pressure sensor, a pump, and the like for measuring blood pressure by an oscillometric method, but the present invention is not limited thereto. In addition, for example, a triaxial acceleration sensor can be adopted as the position detection unit, but other unit may be used as long as at least the position of the device on the vertical axis (that is, the height at which the device is positioned) can be detected.

In addition, in the above description, the “input unit” can be, for example, an operation button provided in the biological information measurement device, but is not limited thereto. For example, a device configuration can be assumed in which measurement is started in a case where a measurement start instruction signal is received from another instrument via communication, and in this case, communication unit functions as the input unit.

According to such a configuration, the collective measurement of the blood pressure and the electrocardiographic waveform can be performed on condition that an electrode contact state suitable for the measurement of the electrocardiographic waveform is obtained by one (measurement start) operation. Therefore, the electrocardiographic waveform is not measured in a state where the electrocardiographic waveform is not correctly in contact with the electrode for electrocardiographic waveform measurement, the measurement does not include the noise caused by the operation for correcting the posture immediately after the start of the measurement, and two types of biological information of the blood pressure value and the electrocardiographic waveform can be collectively measured accurately and easily.

The second correctness determination unit may determine, on the basis of an output of the electrode contact state detection unit, correctness as to whether or not the human body stably is stably in contact with the plurality of electrodes when a determination result of the first correctness determination unit indicates correctness.

With such a configuration, the blood pressure and the electrocardiographic waveform are collectively measured on condition that the posture is suitable for measuring the blood pressure and the electrocardiographic waveform, and more accurate blood pressure measurement can be performed.

In addition, the controller further includes a third correctness determination unit which determines, on the basis of an output of the position detection unit, correctness as to whether or not a wrist of the human body on which the device is worn is positioned at a height within a second predetermined range when a determination result of the second correctness determination unit indicates correctness, and the collective measurement control unit performs control to collectively execute the blood pressure measurement of the human body by the blood pressure measurement unit and the measurement of the electrocardiographic waveform of the human body by the electrocardiogram measurement unit in a case where determination results of the second correctness determination unit and the third correctness determination unit indicate correctness.

With the configuration as described above, the height of the device is roughly adjusted first to prevent the contact with the electrode in an inappropriate posture, and after confirming that the contact state with the electrode is stable, precise height adjustment can be performed again. Accordingly, it is possible to more reliably guide the user to an appropriate posture.

In addition, a height within the second predetermined range may be set to be substantially same as a height of a heart of the human body. Such a height is suitable for the blood pressure measurement. In addition, the biological information measurement device may be a wristwatch-type wearable device.

In addition, the biological information measurement device may further include an output unit,
in which the controller may further include a fourth correctness determination unit which determines whether or not the human body has been in contact with the plurality of electrodes before the correctness determination by the first correctness determination unit is performed, and an electrode preceding contact notification unit which, in a case where a determination result of the fourth correctness determination unit indicates correctness, notifies the determination result via the output unit.

In a case where the contact with the electrode has been made before the first correctness determination is performed, even if the results of the first correctness determination and the second correctness determination indicate correctness, there is a possibility that the posture is actually inappropriate, for example, a palm faces downward. In this regard, with the above-described configuration, it is possible to notify the user that there is a possibility of such a posture, and on the basis of the information, the user can take measures such as performing the measurement (or collective measurement start determination) again.

In addition, the present invention can also be regarded as a control method of a device as follow. That is, provided is a control method of a biological information measurement device which is used by being worn on a wrist of a human body and includes
a blood pressure measurement unit configured to measure a blood pressure of the human body,
an input unit configured to receive an instruction to start blood pressure measurement of the human body,
an electrocardiogram measurement unit which includes a plurality of electrodes and is configured to measure an electrocardiographic waveform of the human body,
an electrode contact state detection unit configured to detects a contact state of the human body with the plurality of electrodes, and
a position detection unit configured to detects a position of the device,
the control method including:
a start instruction reception step which receives an instruction to start blood pressure measurement of the human body;
a first correctness determination step which is executed after the start instruction reception step and determines, on the basis of an output of the position detection unit, correctness as to whether or not a wrist of the human body on which the device is worn is positioned at a height within a first predetermined range;
a second correctness determination step which determines, on the basis of an output of the electrode contact state detection unit, correctness as to whether or not the human body is stably in contact with the plurality of electrodes; and
a collective measurement step which collectively executes blood pressure measurement of the human body by the blood pressure measurement unit and measurement of an electrocardiographic waveform of the human body by the electrocardiogram measurement unit, on condition that at least a determination result in the second correctness determination step indicates correctness.

Note that in the control method, when a determination result in the first correctness determination step indicates correctness, the second correctness determination step may be executed.

In addition, the control method may further include a third correctness determination step which determines, on the basis of an output of the position detection unit, correctness as to whether or not a wrist of the human body on which the device is worn is positioned at a height within a second predetermined range when a determination result in the second correctness determination step indicates correctness,
in which in the collective measurement step, the blood pressure measurement of the human body by the blood pressure measurement unit and the measurement of the electrocardiographic waveform of the human body by the electrocardiogram measurement unit may be collectively performed in a case where a determination result in the third correctness determination step indicates correctness.

In addition, the biological information measurement device may further include an output unit,
the control method of the biological information measurement device may further include:
a fourth correctness determination step which determines correctness as to whether or not the human body has been in contact with the plurality of electrodes before the correctness determination in the first correctness determination step is performed; and
an electrode preceding contact notification step which, in a case where a determination result in the fourth correctness determination step indicates correctness, notifies the determination result via the output unit.

In addition, the present invention can also be regarded as a program for causing the biological information measurement device to execute the above-described method, and a computer-readable recording medium in which such a program is non-transitory recorded.

Note that each of the above-described configurations can be combined with each other to constitute the present invention as long as no technical contradiction occurs.

According to the present invention, it is possible to provide a technique for accurately measuring blood pressure and an electrocardiographic waveform in a portable biological information measurement device capable of measuring the blood pressure and the electrocardiographic waveform.

### BRIEF DESCRIPTION OF DRAWINGS

FIG. 1A is a schematic view illustrating an appearance of a biological information measurement device according to a first embodiment. FIG. 1B is an explanatory view illustrating a state when the biological information measurement device of the first embodiment is worn.

FIG. 2 is a functional block diagram illustrating a functional configuration of the biological information measurement device according to the first embodiment.

FIG. 3A is a first view illustrating an example of an image output in the biological information measurement device of the first embodiment. FIG. 3B is a second view illustrating an example of an image output in the biological information measurement device of the first embodiment. FIG. 3C is a third view illustrating an example of an image output in the biological information measurement device of the first embodiment. FIG. 3D is a fourth view illustrating an example of an image output in the biological information measurement device of the first embodiment.

FIG. 4 is a flowchart illustrating a part of processing performed in the biological information measurement device of the first embodiment.

FIG. 5 is a functional block diagram illustrating a functional configuration of a biological information measurement device according to a second embodiment.

FIG. 6 is a flowchart illustrating a part of processing performed in the biological information measurement device of the second embodiment.

FIG. 7 is an explanatory diagram regarding the processing executed by the biological information measurement device according to the second embodiment.

FIG. 8 is a functional block diagram illustrating a functional configuration of a biological information measurement device according to a third embodiment.

FIG. 9 is a flowchart illustrating a part of processing performed in the biological information measurement device of the third embodiment.

FIG. 10 is an explanatory diagram for explaining a problem in the conventional technique.

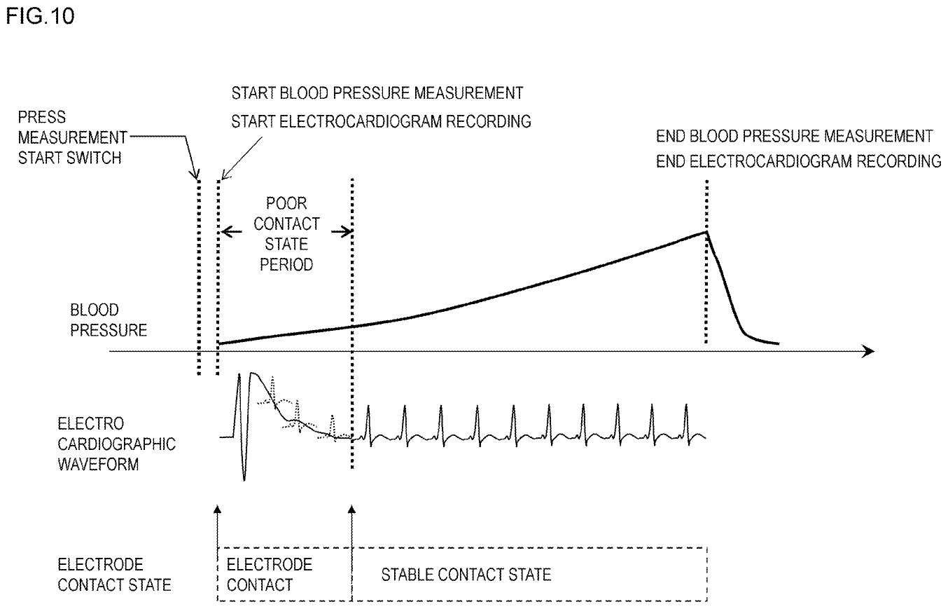

### DESCRIPTION OF EMBODIMENTS

### First Embodiment

Hereinafter, specific embodiments of the present invention will be described on the basis of the drawings. However, the dimensions, materials, shapes, relative arrangements, and the like of the components described in this embodiment are not intended to limit the scope of the present invention only to them unless otherwise specified.

### (Overall Configuration of Device)

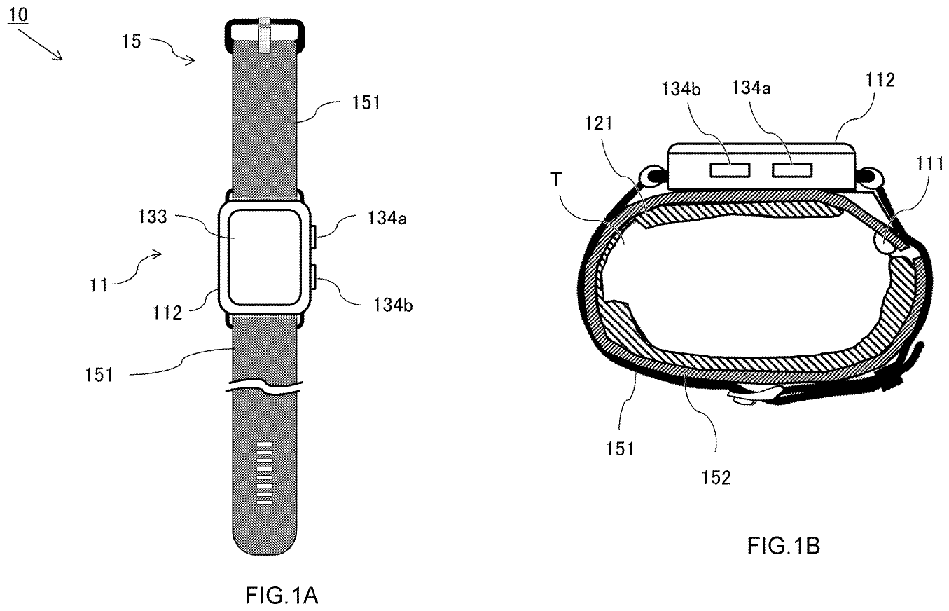

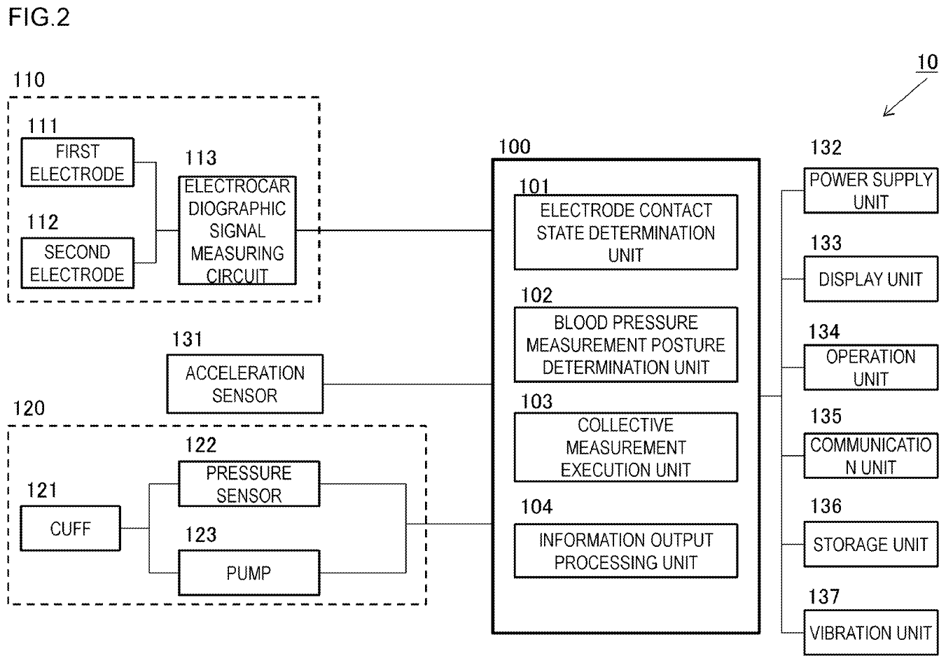

FIG. 1A is a schematic view illustrating an external configuration of a biological information measurement device 10 according to the present embodiment. FIG. 1B is an explanatory view illustrating a state when the biological information measurement device 10 according to the present embodiment is worn on a wrist T. FIG. 2 is a functional block diagram illustrating a functional configuration of the biological information measurement device 10 according to the present embodiment.

As illustrated in FIGS. 1A, 1B, and 2, the biological information measurement device 10 is schematically a wristwatch-type wearable device including a main body 11 and a belt portion 15, and can measure a blood pressure value and an electrocardiographic waveform in the state of being worn on the wrist T of a human body.

The main body 11 includes a display unit 133 (for example, a liquid crystal display or the like can be adopted), operation buttons 134a, 134b, a bezel functioning as second electrode 112, an acceleration sensor 131, and the like. Note that any one of the operation buttons 134a and 134b functions as a measurement start button for starting blood pressure measurement. In addition, the acceleration sensor 131 corresponds to position detection means according to the present invention, and detects the position/posture of the biological information measurement device 10.

In addition, as illustrated in FIG. 2, the main body 11 includes a control unit 100, an electrocardiographic signal measurement unit 110, a blood pressure measurement unit 120, a power supply unit 132, a display unit 133, an operation unit 134, a communication unit 135, a storage unit 136, and a vibration unit 137 as functional configurations thereof. These functional configurations will be described later.

In addition, the belt portion 15 includes a cuff 121 for compressing an artery in the wrist T, a curler 152 for supporting the cuff 121, a first electrode 111, and a belt 151 for fixing the biological information measurement device 10 to the wrist T. For example, the belt 151 includes a parent-side band and a tip-side band, and can adopt a shape of a type in which the tip-side band is fixed by a buckle of the parent-side band. However, the belt may have any configuration as long as the biological information measurement device 10 can be appropriately fixed to the wrist T. For example, it is also possible to adopt a configuration in which fixing is performed by a hook-and-loop fastener.

### (Functional Configurations of Main Body)

Next, the functional configurations of the main body 11 will be described. The control unit 100 controls the entire biological information measurement device 10 including the electrocardiographic signal measurement unit 110, the blood pressure measurement unit 120, and the like. In addition, the control unit 100 includes functional units of an electrode contact state determination unit 101, a blood pressure measurement posture determination unit 102, a collective measurement execution unit 103, and an information output processing unit 104, and reads and executes a program from the storage unit 136 described later, thereby controlling each configuration of the biological information measurement device 10 to realize the functional units which fulfill these predetermined purposes. Note that the control unit 100 includes a processor such as a central processing unit (CPU) in terms of hardware.

The electrocardiographic signal measurement unit 110 includes the first electrode 111, the second electrode 112, and an electrocardiographic signal measuring circuit 113, and measures an electrocardiographic signal of a user on the basis of a potential difference between the first electrode 111 and the second electrode 112 in contact with a human body surface (specifically, the wrist of one hand and the finger of the other hand) (by so-called I induction). In addition, the electrocardiographic signal measuring circuit 113 also detects a contact state of the skin surface of the user with the first electrode 111 and the second electrode 112. That is, the electrocardiographic signal measuring circuit 113 in the present embodiment also serves as electrode contact state detection means according to the present invention. Note that in addition, the electrocardiographic signal measurement unit 110 also includes an AD conversion circuit, an amplifier, a filter, and the like (not illustrated), but since these are configured by known techniques, the description thereof is omitted.

The blood pressure measurement unit 120 includes the cuff 121, the pressure sensor 122, and a pump 123, and measures the blood pressure of the user by a so-called oscillometric method. The blood pressure measurement by the oscillometric method is a well-known technique, and thus a detailed description thereof will be omitted.

The power supply unit 132 includes a battery (not illustrated) which supplies power necessary for operating the device. For example, the battery may be a secondary battery such as a lithium ion battery, or may be a primary battery.

The display unit 133 includes a display device such as a liquid crystal display, and displays, on the display device, various types of information including guide information regarding the operation of the device. Note that the display unit 133 may further include an LED indicator or the like. In addition, the operation unit 134 includes operation buttons 134a and 134b, and receives the input operation of the user via these buttons. Note that the operation unit 134 can also receive an input of a user operation by receiving an input signal from another electronic instrument via the communication unit 135 described later.

The communication unit 135 includes an antenna (not illustrated) for wireless communication, and performs information communication with another electronic instrument such as an information processing terminal by, for example, BLE communication. Note that a terminal for wired communication may be provided.

The storage unit 136 includes a main storage device (not illustrated) such as a random access memory (RAM), and stores various types of information such as an application program, a measured electrocardiographic waveform, a blood pressure, and guide information. In addition to the RAM, a long-term storage medium such as a flash memory may be provided. In addition, a measurement result such as electrocardiographic waveform data and a measured blood pressure value is stored.

The vibration unit 137 includes a vibrator (not illustrated) including a small motor or the like, and generates vibration in a predetermined pattern set for each guidance content. Accordingly, it is possible to notify the user of predetermined guidance information corresponding to the pattern.

Next, each functional unit included in the control unit 100 will be described. The electrode contact state determination unit 101 determines, on the basis of the output of the electrocardiographic signal measuring circuit 113, whether or not the user is stably in contact with the first electrode 111 and the second electrode 112. Whether or not the contact is stable can be distinguished by an arbitrary index, but for example, evaluation may be performed by using information such as the baseline fluctuation of the electrocardiographic waveform and the posture fluctuation of the device based on the output of the acceleration sensor 131.

On the basis of the output of the acceleration sensor 131, the blood pressure measurement posture determination unit 102 determines correctness as to whether or not the wrist of the user in the state of wearing the device is positioned at a height within a predetermined range, more specifically, whether or not the wrist is positioned at a height substantially equal to the height of the heart. In addition, it may be determined whether or not the height is continuously maintained.

On the basis of the outputs of the electrode contact state determination unit 101 and the blood pressure measurement posture determination unit 102, the collective measurement execution unit 103 performs control to collectively execute the measurement of the blood pressure by the blood pressure measurement unit 120 and the measurement of the electrocardiographic waveform in a case where these determination results are both correct. Note that here, the measurement of the electrocardiographic waveform means recording, as waveform data, the electrocardiographic signal measured by the electrocardiographic signal measurement unit 110. That is, in the present embodiment, electrocardiographic waveform measurement means includes the electrocardiographic signal measurement unit 110 and the storage unit 136.

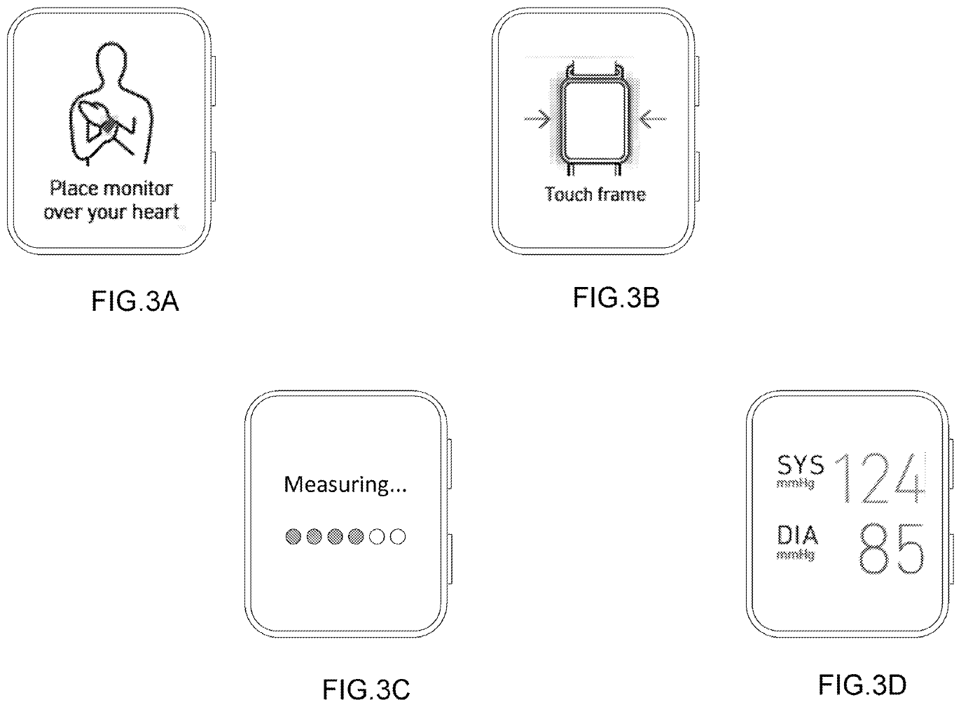

The information output processing unit 104 outputs guide information regarding the use of the device by image display by the display unit 133 and a vibration pattern by the vibration unit 137. Specifically, for example, control of outputting information for guiding a posture for measuring biological information to the user, information for guiding each of the start and end of measurement, and the like is executed. FIGS. 3A to 3D illustrate examples of guide images displayed on the display unit 133.

FIG. 3A is a guide image for guiding raising, to the height of the heart, the wrist on which the device is worn and maintaining the wrist in preparation for measurement. FIG. 3B is a guide image for guiding touching the second electrode 112 of the device in preparation for measurement. FIG. 3C is a guide image for guiding that blood pressure (electrocardiogram) measurement is being performed. FIG. 3D is a guide image illustrating a measurement result after the end of the measurement. Note that each image may be a still image or may be a moving image.

### (Biological Information Measurement Processing)

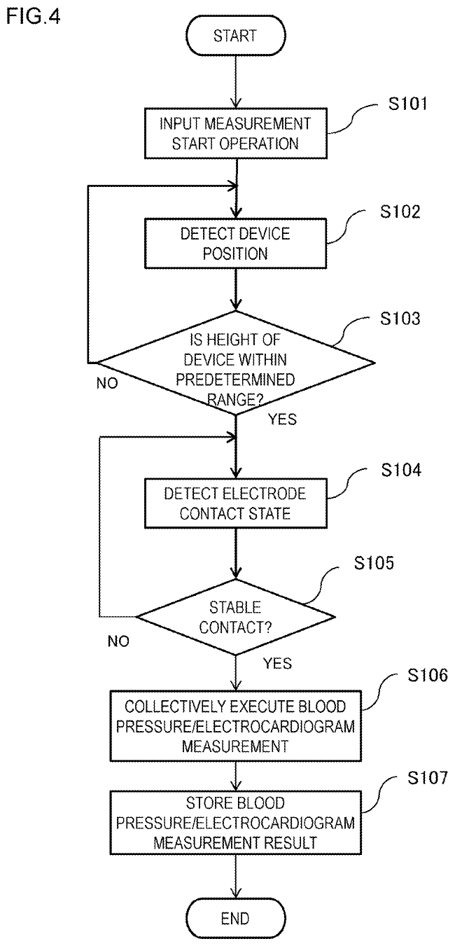

Next, a flow of processing when the biological information measurement device 10 executes measurement of the biological information will be described on the basis of FIG. 4. FIG. 4 is a flowchart illustrating a procedure of processing when the biological information measurement device 10 according to the present embodiment is used to collectively measure the blood pressure and the electrocardiographic waveform.

First, the biological information measurement device 10 receives a blood pressure measurement start operation from the user via the operation unit 134 (S101). The acceleration sensor 131 detects the position/posture of the device (S102), and the blood pressure measurement posture determination unit 102 determines, on the basis of on the output of the acceleration sensor 131, whether or not the height of the biological information measurement device 10 is within a predetermined range (S103). Here, in a case where it is determined that the height of the device is not within the predetermined range, the process returns to step S102, and the determination processing as to whether the height of the device is within the predetermined range is repeated on the basis of the output of the acceleration sensor 131.

On the other hand, in a case where it is determined in step S103 that the height of the device is within the predetermined range, the process proceeds to step S104. In step S104, the electrocardiographic signal measuring circuit 113 detects a contact state of the human body (user) with the first electrode 111 and the second electrode 112 (S104). Then, the electrode contact state determination unit 101 determines, on the basis of the output of the electrocardiographic signal measuring circuit 113, correctness as to whether or not the user is stably in contact with the first electrode 111 and the second electrode 112 (S105). Here, in a case where it is determined that the user is not stably in contact with each electrode, the process returns to step S104, and the subsequent processing is repeated.

On the other hand, when it is determined in step S105 that the user is stably in contact with each electrode, the collective measurement execution unit 103 performs control to collectively execute the measurement of the blood pressure and the measurement of the electrocardiographic waveform by the blood pressure measurement unit 120 (S106). Then, when the measurement of the blood pressure is ended, the measurement of the electrocardiographic waveform (that is, recording of waveform data) is also ended simultaneously, the measurement result is stored in the storage unit 136 (S107), and this routine is temporarily ended.

Note that the information output processing unit 104 may output the guide information at an appropriate timing of the above flow. For example, prior to step S102, a guide that the wrist on which the device is worn is to be raised to the height of the heart and maintained may be provided by displaying the guide image illustrated in FIG. 3A on the display unit 133 or by a predetermined vibration pattern by the vibration unit 137. In addition, after step S106, an image (see FIG. 3D) indicating the measurement result may be displayed on the display unit 133.

According to the biological information measurement device 10 according to the present embodiment as described above, the blood pressure and the electrocardiographic waveform are collectively measured by maintaining, at a height suitable for blood pressure measurement, a site where the blood pressure is measured in the state where the device is worn (that is, the position of the device) and establishing a situation of the stable contact with the electrode. Therefore, it is possible to prevent the measurement from being performed in an inappropriate posture or situation for performing the measurement, and it is possible to obtain a measurement result with high accuracy for both the blood pressure and the electrocardiographic waveform.

### (Modification)

Note that, in the flow of the biological information measurement processing described above, in a case where it is determined in step S103 that the height of the device is not within the predetermined range, the process returns to step S102 and does not proceed to step S104 until the condition is satisfied, but other processing can be performed. For example, in a case where it is determined that the height of the device is not within the predetermined range, that information may be stored in the storage unit 136, and then the process may proceed to step S104. That is, in the present modification, when the determination result of the electrode contact state determination unit 101 indicates correctness, the collective measurement execution unit 103 performs control to collectively execute the measurement of the blood pressure and the measurement of the electrocardiographic waveform by the blood pressure measurement unit 120. In this manner, it is possible to prevent that the measurement is not started indefinitely unless the correct posture is taken, and to store that there is a doubt in the accuracy of the measured blood pressure value and obtain at least an electrocardiographic waveform with high accuracy.

### Second Embodiment

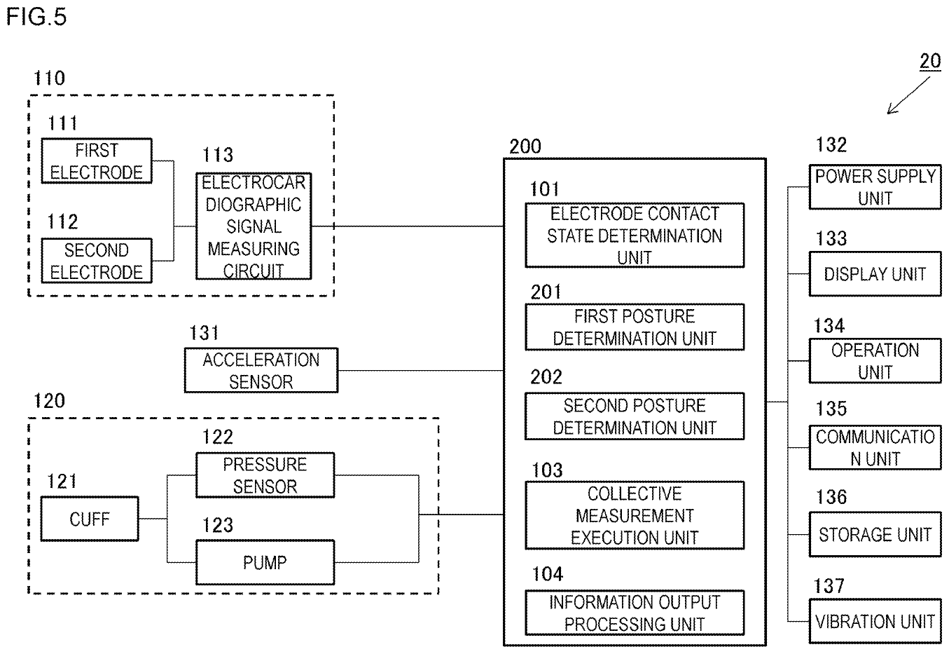

Next, another embodiment of the present invention will be described on the basis of FIGS. 5 to 7. A biological information measurement device 20 according to the present embodiment has almost the same configuration as the biological information measurement device 10 according to the first embodiment. Therefore, the same configurations and processing as those in the first embodiment are denoted by the same reference numerals, and detailed description thereof will be omitted.

FIG. 5 is a functional block diagram illustrating a functional configuration of the biological information measurement device 10 according to the present embodiment. As illustrated in FIG. 5, the biological information measurement device 20 according to the present embodiment is partially different from the biological information measurement device 10 in the functional units included in the control unit 200. Specifically, the control unit 200 includes a first posture determination unit 201 and a second posture determination unit 202 instead of the blood pressure measurement posture determination unit 102.

Both the first posture determination unit 201 and the second posture determination unit 202 are functional units that determine whether or not the height of the biological information measurement device 20 is within a predetermined range, but the threshold value of the second posture determination unit 202 is set more strictly than that of the first posture determination unit 201.

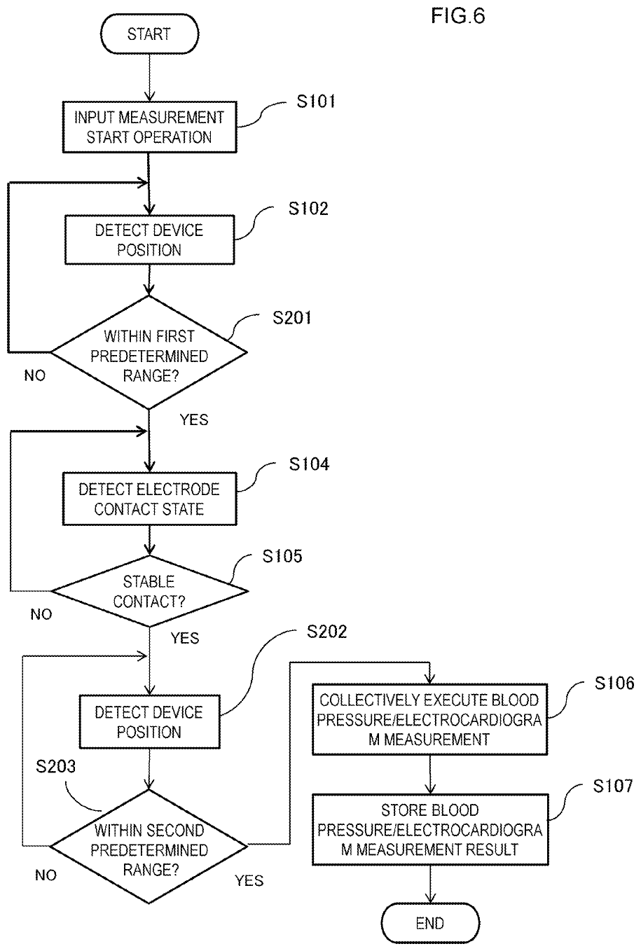

Next, a flow of processing when the biological information measurement device 20 executes measurement of the biological information is executed on the basis of FIG. 6. As illustrated in FIG. 6, also in the present embodiment, the flow of processing is substantially the same as that in the first embodiment. When the measurement start operation is input (S101), the process proceeds to step S102, and then the first posture determination unit 201 determines whether or not the device is at a height within a first predetermined range (S201). Here, whether or not the posture is an appropriate posture for measurement is roughly determined on the basis of the height at which the device is positioned. In a case where it is determined in step S201 that the height is not within the first predetermined range, the process returns to step S102, and the determination processing as to whether the height of the device is within the predetermined range is repeated on the basis of the output of the acceleration sensor 131.

On the other hand, in a case where it is determined in step S201 that the height is within the first predetermined range, the process proceeds to step S104. Thereafter, in a case where it is determined in step S105 that both electrodes are stably touched, the position/posture of the device is detected again (S202), and whether or not the height of the device is within a second predetermined range is determined on the basis of the output of the acceleration sensor 131 (S203). Here, in a case where it is determined that the height is not within the second predetermined range, the process returns to step S202, and the processing of determining whether or not the height of the device is within the second predetermined range is repeated on the basis of the output of the acceleration sensor 131.

On the other hand, when it is determined in step S203 that the height of the device is within the second predetermined range, the process proceeds to step S106, and the collective measurement processing of the blood pressure and the electrocardiographic waveform is performed. Since subsequent processing is the same as that in the first embodiment, the description thereof will be omitted.

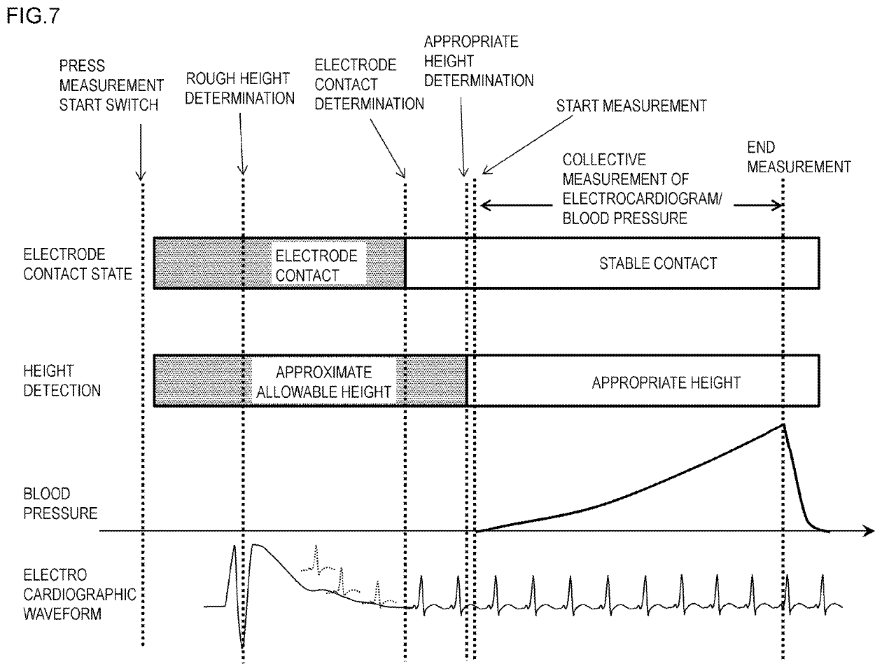

FIG. 7 is an explanatory diagram illustrating a relationship between the processing executed by the biological information measurement device 20 according to the present embodiment, and the device position (posture) and the electrode contact state in time series together with the measured biological information. As illustrated in FIG. 7, according to the biological information measurement device 20 of the present embodiment, after the measurement start operation input, first, it is roughly determined based on a first threshold value whether the posture for blood pressure measurement is taken. Thereafter, processing of determining whether or not the contact state of the electrode is stable is performed, and then when it is determined that the contact state is stable, processing of determining, with a relatively strict threshold value, whether or not the device is positioned at a correct height suitable for blood pressure measurement is further executed. Then, when it is determined in the second height determination processing that the device is positioned at an appropriate height, the blood pressure and the electrocardiographic waveform are collectively measured.

According to such a configuration, the height of the device is roughly adjusted first to prevent the contact with the electrode in an inappropriate posture, and after confirming that the contact state with the electrode is stable, precise height adjustment can be performed again, whereby the user can be guided more reliably to an appropriate posture.

### Third Embodiment

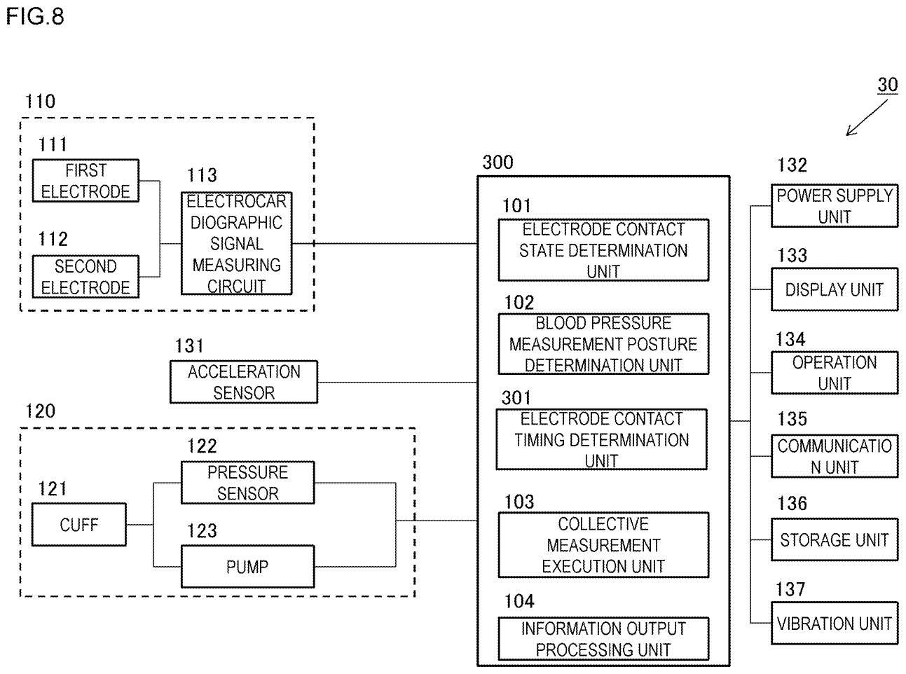

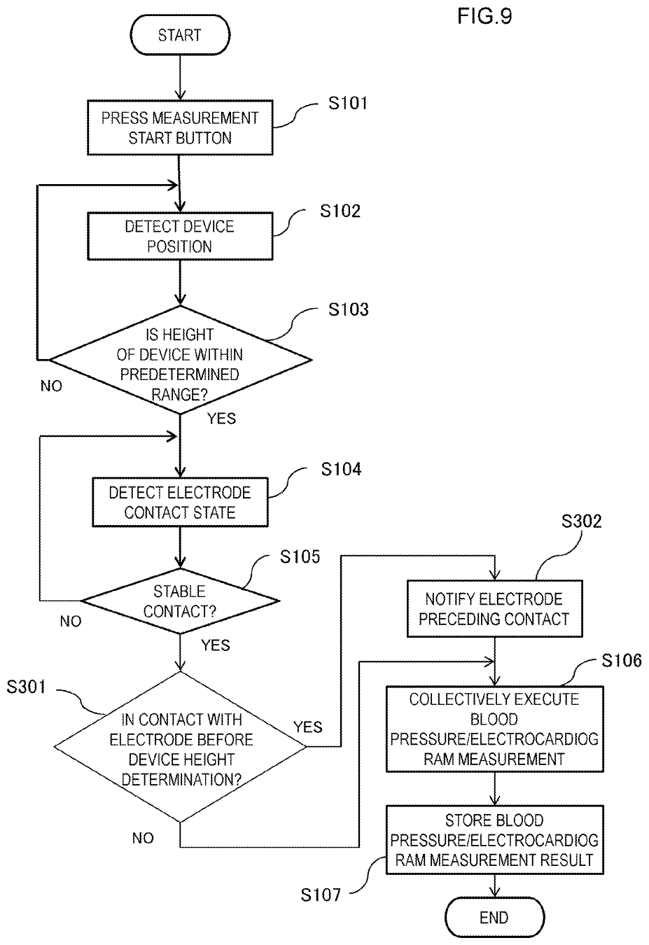

Next, still another embodiment of the present invention will be described on the basis of FIGS. 8 and 9. A biological information measurement device 30 according to the present embodiment has almost the same configuration as the biological information measurement device 10 according to the first embodiment. Therefore, the same configurations and processing as those in the first embodiment are denoted by the same reference numerals, and detailed description thereof will be omitted.

FIG. 8 is a functional block diagram illustrating a functional configuration of the biological information measurement device 30 according to the present embodiment. As illustrated in FIG. 8, the biological information measurement device 30 according to the present embodiment is partially different from the biological information measurement device 10 in the functional units included in the control unit 300. Specifically, the control unit 300 further includes an electrode contact timing determination unit 301.

On the basis of the output of the electrocardiographic signal measuring circuit 113, the electrode contact timing determination unit 301 determines whether or not the user has been in contact with the first electrode 111 and the second electrode 112 before the execution of the height determination by the blood pressure measurement posture determination unit 102.

Then, when the user has been in contact with the first electrode 111 and the second electrode before the execution of the height determination by the blood pressure measurement posture determination unit 102, the information output processing unit 104 outputs guide information notifying the user of the fact by image display on the display unit 133 or a vibration pattern by the vibration unit 137. That is, in the present embodiment, the information output processing unit 104 corresponds to an electrode preceding contact notification unit.

Next, a flow of processing when the biological information measurement device 30 executes measurement of biological information is executed on the basis of FIG. 9. As illustrated in FIG. 9, also in the present embodiment, the flow of processing is substantially the same as that in the first embodiment, and is different in that the processing of steps S301 and S302 is added.

In the biological information measurement device 30 according to the present embodiment, when it is determined in step S105 that the user is stably in contact with both electrodes, the electrode contact timing determination unit 301 determines whether or not the user has been in contact with the first electrode 111 and the second electrode before the execution of the height determination (S301). Here, when it is determined that the second electrode is not in contact before the execution of the height determination, the process proceeds to step S106, and the blood pressure and the electrocardiographic waveform are collectively measured. On the other hand, in a case where it is determined in step S301 that both electrodes have been in contact before the execution of the height determination, the information output processing unit 104 outputs a notification of the result to the user (S302). However, thereafter, the process proceeds to S106, and the blood pressure and the electrocardiographic waveform are collectively measured. Since subsequent processing is the same as that in the first embodiment, the description thereof will be omitted.

In a case where both electrodes have been in contact with each other before the execution of the height determination, even when the determination result indicates appropriateness in steps S103 and S105, there is a possibility that the posture is actually inappropriate, for example, the palm is directed downward. In this regard, with the above-described configuration, it is possible to notify the user that there is a possibility of an inappropriate posture, and on the basis of the information, the user can take measures such as performing the measurement (or collective measurement start determination) again.

### Others

The description of the above-described embodiments is merely illustrative of the present invention, and the present invention is not limited to the above-described specific forms. The present invention can be variously modified and combined within the scope of the technical idea. For example, in the above-described embodiment, it has been described that the guide image is displayed on the display unit 133, but the guide image may be output to an external instrument connected via the communication unit 135. In addition, the measured biological information may be streamed and transmitted to an external electronic instrument including a storage area via the communication unit 135.

In addition, the input means that receives an instruction to start the blood pressure measurement is not limited to the operation buttons 134a and 134b in each of the above-described embodiments. For example, the blood pressure measurement may be started by receiving a measurement start signal from an external electronic instrument via the communication unit 135. That is, the input means in the present invention is not limited to the configuration such as the operation button.

1. A biological information measurement device used by being worn on a wrist of a human body, the biological information measurement device comprising:
a blood pressure measurement unit configured to measure a blood pressure of the human body;
an input unit configured to receive an instruction to start blood pressure measurement of the human body;
an electrocardiographic waveform measurement unit which includes a plurality of electrodes and is configured to measure an electrocardiographic waveform of the human body;
an electrode contact state detection unit configured to detect a contact state of the human body with the plurality of electrodes;
a position detection unit configured to detect a position of the device; and
a controller configured to control the electrocardiographic waveform measurement unit and the blood pressure measurement unit,
wherein the controller includes
a first correctness determination unit which determines, on a basis of an output of the position detection unit, correctness as to whether or not a wrist of the human body on which the device is worn is positioned at a height within a first predetermined range after receiving an instruction to start blood pressure measurement of the human body via the input unit,
a second correctness determination unit which determines, on a basis of an output of the electrode contact state detection unit, correctness as to whether or not the human body is stably in contact with the plurality of electrodes when a determination result of the first correctness determination unit indicates correctness,
a third correctness determination unit which determines, on a basis of an output of the position detection unit, correctness as to whether or not the wrist of the human body on which the device is worn is positioned at a height within a second predetermined range when a determination result of the second correctness determination unit indicates correctness, and
a collective measurement control unit which performs control to collectively execute blood pressure measurement of the human body by the blood pressure measurement unit and measurement of an electrocardiographic waveform of the human body by the electrocardiographic waveform measurement unit, on condition that determination result of the second correctness determination unit and the third correctness determination unit indicates correctness.

2. The biological information measurement device according to claim 1, wherein a height within the second predetermined range is set to be substantially same as a height of a heart of the human body.

3. A biological information measurement device used by being worn on a wrist of a human body, the biological information measurement device comprising:
a blood pressure measurement unit configured to measure a blood pressure of the human body;
an input unit configured to receive an instruction to start blood pressure measurement of the human body;
an electrocardiographic waveform measurement unit which includes a plurality of electrodes and is configured to measure an electrocardiographic waveform of the human body;
an electrode contact state detection configured to detect a contact state of the human body with the plurality of electrodes;
an position detection unit configured to detect a position of the device;
a controller configured to control the electrocardiographic waveform measurement unit and the blood pressure measurement unit; and
an output unit,
wherein the controller includes
a first correctness determination unit which determines, on a basis of an output of the position detection unit, correctness as to whether or not a wrist of the human body on which the device is worn is positioned at a height within a first predetermined range after receiving an instruction to start blood pressure measurement of the human body via the input unit,
a second correctness determination unit which determines, on a basis of an output of the electrode contact state detection unit, correctness as to whether or not the human body is stably in contact with the plurality of electrodes,
a collective measurement control unit which performs control to collectively execute blood pressure measurement of the human body by the blood pressure measurement unit and measurement of an electrocardiographic waveform of the human body by the electrocardiographic waveform measurement unit, on condition that at least a determination result of the second correctness determination unit indicates correctness,
a fourth correctness determination unit which determines whether or not the human body has been in contact with the plurality of electrodes before the correctness determination by the first correctness determination unit is performed, and
an electrode preceding contact notification unit which, in a case where a determination result of the fourth correctness determination unit indicates correctness, notifies the determination result via the output unit.

4. The biological information measurement device according to claim 1, wherein the biological information measurement device is a wristwatch-type wearable device.

5. A control method of a biological information measurement device which is used by being worn on a wrist of a human body and includes
a blood pressure measurement unit configured to measure a blood pressure of the human body,
an input unit configured to receive an instruction to start blood pressure measurement of the human body,
an electrocardiographic waveform measurement unit which includes a plurality of electrodes and is configured to measure an electrocardiographic waveform of the human body,
an electrode contact state detection unit configured to detect a contact state of the human body with the plurality of electrodes, and
a position detection unit configured to detect a position of the device,
the control method comprising:
a start instruction reception step which receives an instruction to start blood pressure measurement of the human body;
a first correctness determination step which is executed after the start instruction reception step and determines, on a basis of an output of the position detection unit, correctness as to whether or not a wrist of the human body on which the device is worn is positioned at a height within a first predetermined range;
a second correctness determination step which determines, on a basis of an output of the electrode contact state detection unit, correctness as to whether or not the human body is stably in contact with the plurality of electrodes;
a third correctness determination step which determines, on the basis of an output of the position detection unit, correctness as to whether or not the wrist of the human body on which the device is worn is positioned at a height within a second predetermined range when a determination result in the second correctness determination step indicates correctness; and
a collective measurement step which collectively executes blood pressure measurement of the human body by the blood pressure measurement unit and measurement of an electrocardiographic waveform of the human body by the electrocardiographic waveform measurement unit, on condition that determination result in the third correctness determination step indicates correctness.

6. The control method of the biological information measurement device according to claim 5, wherein a height within the second predetermined range is set to be substantially same as a height of a heart of the human body.

7. A control method of a biological information measurement device which is used by being worn on a wrist of a human body and includes
a blood pressure measurement unit configured to measure a blood pressure of the human body,
an input unit configured to receive an instruction to start blood pressure measurement of the human body,
an electrocardiographic waveform measurement unit which includes a plurality of electrodes and is configured to measure an electrocardiographic waveform of the human body,
an electrode contact state detection unit configured to detect a contact state of the human body with the plurality of electrodes,
a position detection unit configured to detect a position of the device, and
an output unit,
the control method comprising:
a start instruction reception step which receives an instruction to start blood pressure measurement of the human body;
a first correctness determination step which is executed after the start instruction reception step and determines, on a basis of an output of the position detection unit, correctness as to whether or not a wrist of the human body on which the device is worn is positioned at a height within a first predetermined range;
a second correctness determination step which determines, on a basis of an output of the electrode contact state detection unit, correctness as to whether or not the human body is stably in contact with the plurality of electrodes;
a collective measurement step which collectively executes blood pressure measurement of the human body by the blood pressure measurement unit and measurement of an electrocardiographic waveform of the human body by the electrocardiographic waveform measurement unit, on condition that at least a determination result in the second correctness determination step indicates correctness;
a fourth correctness determination step which determines correctness as to whether or not the human body has been in contact with the plurality of electrodes before the correctness determination in the first correctness determination step is performed; and
an electrode preceding contact notification step which, in a case where a determination result in the fourth correctness determination step indicates correctness, notifies the determination result via the output unit.

8. A non-transitory computer readable medium storing a program for causing a biological information measurement device to execute each step of the control method according to claim 5.
